SEQUENCE LISTING (23 sequences)

SEQ ID NO: 1 - protein, 522 aa, Homo sapiens
MAVNKGLTLLDGDLPEQENVLQRVLQLPVVSGTCECFQKTYTSTKEAHPLVASVCNAYEKGVQSASSLAAWSMEPVVRRLSTQFTAANELACRGLDHLEEKIPALQYPPEKIASELKDTISTRLRSARNSISVPIASTSDKVLGAALAGCELAWGVARDTAEFAANTRAGRLASGGADLALGSIEKVVEYLLPPDKEESAPAPGHQQAQKSPKAKPSLLSRVGALTNTLSRYTVQTMARALEQGHTVAMWIPGVVPLSSLAQWGASVAMQAVSRRRSEVRVPWLHSLAAAQEEDHEDQTDTEGEDTEEEEELETEENKFSEVAALPGPRGLLGGVAHTLQKTLQTTISAVTWAPAAVLGMAGRVLHLTPAPAVSSTKGRAMSLSDALKGVTDNVVDTVVHYVPLPRLSLMEPESEFRDIDNPPAEVERREAERRASGAPSAGPEPAPRLAQPRRSLRSAQSPGAPPGPGLEDEVATPAAPRPGFPAVPREKPKRRVSDSFFRPSVMEPILGRTHYSQLRKKS

SEQ ID NO: 2 - protein, 49 aa, Homo sapiens
LQLPVVSGTCECFQKTYTSTKEAHPLVASVCNAYEKGVQSASSLAAWSM

SEQ ID NO: 3 - protein, 49 aa, Homo sapiens
PLVASVCNAYEKGVQSASSLAAWSMEPVVRRLSTQFTAANELACRGLDH

SEQ ID NO: 4 - protein, 49 aa, Homo sapiens
ASVAMQAVSRRRSEVRVPWLHSLAAAQEEDHEDQTDTEGEDTEEEEELE

SEQ ID NO: 5 - protein, 49 aa, Homo sapiens
HLEEKIPALQYPPEKIASELKDTISTRLRSARNSISVPIASTSDKVLGA

SEQ ID NO: 6 - protein, 49 aa, Homo sapiens
STRLRSARNSISVPIASTSDKVLGAALAGCELAWGVARDTAEFAANTRA

SEQ ID NO: 7 - protein, 49 aa, Homo sapiens
MAVNKGLTLLDGDLPEQENVLQRVLQLPVVSGTCECFQKTYTSTKEAHP

SEQ ID NO: 8 - protein, 49 aa, Homo sapiens
MEPVVRRLSTQFTAANELACRGLDHLEEKIPALQYPPEKIASELKDTIS

SEQ ID NO: 9 - protein, 49 aa, Homo sapiens
AALAGCELAWGVARDTAEFAANTRAGRLASGGADLALGSIEKVVEYLLP

SEQ ID NO: 10 - protein, 49 aa, Homo sapiens
AGRLASGGADLALGSIEKVVEYLLPPDKEESAPAPGHQQAQKSPKAKPS

SEQ ID NO: 11 - protein, 49 aa, Homo sapiens
PPDKEESAPAPGHQQAQKSPKAKPSLLSRVGALTNTLSRYTVQTMARAL

SEQ ID NO: 12 - protein, 49 aa, Homo sapiens
SLLSRVGALTNTLSRYTVQTMARALEQGHTVAMWIPGVVPLSSLAQWGA

SEQ ID NO: 13 - protein, 49 aa, Homo sapiens
LEQGHTVAMWIPGVVPLSSLAQWGASVAMQAVSRRRSEVRVPWLHSLAA

SEQ ID NO: 14 - protein, 49 aa, Homo sapiens
AAQEEDHEDQTDTEGEDTEEEEELETEENKFSEVAALPGPRGLLGGVAH

SEQ ID NO: 15 - protein, 49 aa, Homo sapiens
ETEENKFSEVAALPGPRGLLGGVAHTLQKTLQTTISAVTWAPAAVLGMA

SEQ ID NO: 16 - protein, 49 aa, Homo sapiens
HTLQKTLQTTISAVTWAPAAVLGMAGRVLHLTPAPAVSSTKGRAMSLSD

SEQ ID NO: 17 - protein, 49 aa, Homo sapiens
AGRVLHLTPAPAVSSTKGRAMSLSDALKGVTDNVVDTVVHYVPLPRLSL

SEQ ID NO: 18 - protein, 49 aa, Homo sapiens
DALKGVTDNVVDTVVHYVPLPRLSLMEPESEFRDIDNPPAEVERREAER

SEQ ID NO: 19 - protein, 49 aa, Homo sapiens
LMEPESEFRDIDNPPAEVERREAERRASGAPSAGPEPAPRLAQPRRSLR

SEQ ID NO: 20 - protein, 49 aa, Homo sapiens
RRASGAPSAGPEPAPRLAQPRRSLRSAQSPGAPPGPGLEDEVATPAAPR

SEQ ID NO: 21 - protein, 49 aa, Homo sapiens
RSAQSPGAPPGPGLEDEVATPAAPRPGFPAVPREKPKRRVSDSFFRPSV

SEQ ID NO: 22 - protein, 42 aa, Homo sapiens
RPGFPAVPREKPKRRVSDSFFRPSVMEPILGRTHYSQLRKKS

SEQ ID NO: 23 - protein, 18 aa, Homo sapiens
VMEPILGRTHYSQLRKKS

(19) United States
(12) Patent Application Publication
(10) Pub. No.: US 2024/0019431 A1
(43) Pub. Date: Jan. 18, 2024

(54) AUTOANTIBODIES AS BIOMARKERS FOR LIPODYSTROPHY

(71) Applicants: CZ Biohub SF, LLC, San Francisco, CA (US); The Regents of the University of California, Oakland, CA (US)

(72) Inventors: Mark Anderson, Oakland, CA (US); Joseph L. DeRisi, San Francisco, CA (US); Caleigh Mandel-Brehm, Oakland, CA (US)

(21) Appl. No.: 18/251,926

(22) PCT Filed: Dec. 22, 2021
(86) PCT No.: PCT/US2021/064919

§ 371 Date: May 5, 2023

Related U.S. Application Data
(60) Provisional application No. 63/131,255, filed on Dec. 28, 2020.

(51) Int. Cl. G01N 33/564; C07K 14/47

(52) U.S. Cl. CPC G01N 33/564; C07K 14/4702; G01N 2800/24; G01N 2800/50; G01N 2800/044

(57) ABSTRACT

This disclosure describes methods and compositions for detecting the presence of a Perilipin 1 (PLIN1) autoantibody in a biological sample obtained from a subject that has or is being treated with immune checkpoint inhibitor therapy. Also provided are methods of treating subjects with acquired lipodystrophy.

### CROSS-REFERENCE TO RELATED APPLICATIONS

This application claims the benefit of priority of U.S. Provisional Application No. 63/131,255, filed Dec. 28, 2020. This provisional application is incorporated by reference herein in its entirety for all purposes.

### SEQUENCE LISTING

The instant application contains a Sequence Listing which has been submitted electronically in ASCII format and is hereby incorporated by reference in its entirety. Said ASCII copy, created on Dec. 15, 2021, is named 1277216_seqlist.txt and is 15,352 bytes in size.

### BACKGROUND

Cancer immunotherapies have rapidly evolved in recent years and yielded clinical success in the treatment of .cancers. One of the most widely used immunotherapies include blockade of checkpoint proteins by immune checkpoint inhibitors (ICIs). These drugs work by inhibiting negative regulation of T cells, thus leading to heightened antitumor responses. Though these drugs have improved outcomes for some advanced malignancies, they are not without side-effects. The mechanism of these drugs, non-specifically activating T cells, also leads to immune-mediated damage of tissue or immune-related adverse events (IRAE). IRAEs have been described that affect nearly every organ system. Colitis, various rashes, pneumonitis, hepatitis, encephalopathy, neuropathy, thyroiditis and hypophysitis are some of the wide-ranging adverse effects attributed to ICIs. Recently, lipodystrophy has been described in patients that underwent immune checkpoint blockade therapy.

Lipodystrophy is a clinical phenotype defined by a reduction in subcutaneous fat or adipose tissue that may occur in some parts of the body (partial lipodystrophy), or throughout (generalized lipodystrophy). Patients with partial lipodystrophy may exhibit excess adipose tissue accumulation in other areas of the body. Akin to an excessive gain in fat, excessive fat loss can be damaging to health. The pathological loss of adipocytes, or fat cells, leads to an imbalance of lipid metabolism, storage and signaling, which can manifest as a myriad of clinically severe outcomes, including insulin resistance that may include type II diabetes. Other common manifestations are hypertriglyceridemia, hepatic steatosis which can progress to steatohepatitis, acanthosis nigricans, polycystic ovarian syndrome (PCOS), and eruptive xanthomas (due to severe hypertriglyceridemia). Metabolic derangements are mostly responsible for the serious comorbidities associated with lipodystrophy. As the use of immune checkpoint blockade therapies expands, it will become increasingly important to identify patients at risk for the development of lipodystrophy.

### BRIEF SUMMARY

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim.

In one aspect, provided is a method of identifying a subject that is at risk of developing autoimmune-related lipodystrophy, the method comprising:
(a) contacting a biological sample from the subject with a PLIN1 antigenic polypeptide, or a fragment thereof; and
(b) detecting the presence of binding of the PLIN1 antigenic polypeptide or fragment thereof to PLIN1 autoantibody in the biological sample,
wherein the subject has been previously treated or is being treated with immune checkpoint inhibitor (ICI) therapy.

In another aspect, provided is a method of identifying early evidence of autoimmune-related lipodystrophy in a subject that has been previously treated or is being treated with immune checkpoint inhibitor (ICI) therapy, the method comprising:
(a) contacting a biological sample from the subject with a PLIN1 antigenic polypeptide, or a fragment thereof; and
(b) detecting the presence of binding of the PLIN1 antigenic polypeptide or fragment thereof to PLIN1 autoantibody in the biological sample.

In another aspect, provided is a method of detecting the presence of a Perilipin-1 (PLIN1) autoantibody in a biological sample from a subject that has been previously treated or is being treated with immune checkpoint inhibitor (ICI) therapy, the method comprising:
(a) contacting the biological sample with a PLIN1 antigenic polypeptide, or a fragment thereof; and
(b) detecting the presence of binding of the PLIN1 antigenic polypeptide or fragment thereof to PLIN1 autoantibody in the biological sample.

In some instances, the presence of the binding of the PLIN1 antigenic polypeptide or fragment thereof to the PLIN1 autoantibody in the biological sample indicates that the subject is at risk of developing autoimmune-related lipodystrophy.

In some instances, the subject is suspected to have lipodystrophy or is diagnosed with lipodystrophy.

In some instances, the presence of the binding of the PLIN1 antigenic polypeptide or fragment thereof to the PLIN1 autoantibody in the biological sample indicates that the subject has autoimmune-related lipodystrophy.

In some instances, the subject has autoimmune-related lipodystrophy, and wherein the PLIN1 antigenic polypeptide or fragment thereof binds to said PLIN1 autoantibody in the biological sample.

In some instances, the PLIN1 antigenic polypeptide or fragment thereof is heterologously expressed on the surface of a cell, a phage or a virus.

In some instances, the PLIN1 antigenic polypeptide or fragment thereof is expressed in a phage display or eukaryotic cell display library.

In some instances, the PLIN1 antigenic polypeptide or fragment thereof is an isolated, purified antigenic polypeptide or fragment thereof.

In some instances, the PLIN1 antigenic polypeptide or fragment thereof is an isolated, purified antigenic polypeptide or fragment thereof that is immobilized on a solid carrier.

In some instances, step (b) of detecting the presence of binding of the PLIN1 antigenic polypeptide or fragment thereof to a PLIN1 autoantibody is performed by at least one of immunoprecipitation, microarray analysis, enzyme-linked immunosorbent assay (ELISA), or Western blot analysis.

In some instances, the PLIN1 polypeptide or fragment thereof comprises one or more of the sequences of SEQ ID NOs: 2-6.

In some instances, the PLIN1 antigenic polypeptide comprises the sequence of SEQ ID NO:1.

In some instances, the biological sample is serum, plasma, blood, or urine.

In some instances, the autoimmune-related lipodystrophy is acquired generalized lipodystrophy (AGL) or acquired partial lipodystrophy (APL).

In some instances, the subject has one or more of insulin resistance, low serum leptin, hyperphagia, polydipsia, dyslipidemia, acute pancreatitis, hepatic cirrhosis, proteinuria and renal failure, or polyuria.

In some instances, the subject has one or more of type II diabetes, hypertriglyceridemia, hepatic steatosis, steatohepatitis, acanthosis nigricans, polycystic ovarian syndrome (PCOS), cardiovascular disease, or eruptive xanthomas.

In some instances, the subject has one or more of an autoimmune disease.

In some instances, the autoimmune disease is dermatomyositis, polymyositis, Sjogren's syndrome, rheumatoid arthritis, psoriatic arthritis, ankylosing spondylitis, scleroderma, systemic sclerosis, or systemic lupus erythematosus.

In some instances, the subject has cancer or has had cancer.

In another aspect, provided herein are devices, kits and panels useful for the diagnosis and prognostic assessment of subjects that have been previously treated or is being treated with immune checkpoint inhibitor (ICI) therapy. In some aspects, the devices, kits and panels are used for identifying early evidence of autoimmune-related lipodystrophy in subjects that have been previously treated or is being treated with ICI therapy. In some aspects, the devices, kits and panels are used for assessing the risk for developing autoimmune-related lipodystrophy in subjects that have been previously treated or are being treated with ICI therapy. In some aspect, provided in this disclosure are kits and panels containing one or more PLIN1 polypeptides or antigenic fragments thereof to which PLIN1 autoantibodies can specifically bind. The polypeptide used in the kits and panels is preferably designed such that it is immunogenic, particularly that it binds to PLIN1 autoantibodies from subjects.

Also provided herein is a method of treating a subject having early evidence of lipodystrophy, the method comprising:
(a) detecting the presence PLIN1 autoantibody that binds specifically to a PLIN1 antigenic polypeptide or fragment thereof in a biological sample from a subject, wherein detecting the PLIN1 autoantibody in the biological sample indicates that the subject has lipodystrophy; and
(b) administering to the subject an immunosuppressive therapy and/or a peroxisome proliferator-activated receptor (PPAR) agonist,
wherein the subject has been previously treated or is being treated with immune checkpoint blockade therapy.

Also provided herein is a method of treating a subject having early evidence of lipodystrophy, the method comprising administering to a subject an immunosuppressive therapy and/or a peroxisome proliferator-activated receptor (PPAR) agonist, wherein the subject has been previously treated or is being treated with immune checkpoint blockade therapy.

In some instances, the subject has symptoms of acquired generalized lipodystrophy (AGL) or acquired partial lipodystrophy (APL).

Also provided is a method of treating a subject that is at risk of developing lipodystrophy, the method comprising:
(a) detecting the presence or absence of PLIN1 autoantibody in a biological sample from a subject using a method as provided herein, wherein detecting the PLIN1 autoantibody in the biological sample indicates that the subject is at risk of developing lipodystrophy; and
(b) administering to the subject an immunosuppressive therapy and/or a peroxisome proliferator-activated receptor (PPAR) agonist,
wherein the subject has been previously treated or is being treated with immune checkpoint blockade therapy.

Also provided herein is a method of treating a subject that is at risk of developing lipodystrophy, the method comprising administering to a subject an immunosuppressive therapy and/or a peroxisome proliferator-activated receptor (PPAR) agonist, wherein the subject has been previously treated or is being treated with immune checkpoint blockade therapy.

In some instances, the subject has one or more of insulin resistance, low serum leptin, hyperphagia, polydipsia, dyslipidemia, acute pancreatitis, hepatic cirrhosis, proteinuria and renal failure, or polyuria. In some instances, the subject has one or more of type II diabetes, hypertriglyceridemia, hepatic steatosis, steatohepatitis, acanthosis nigricans, polycystic ovarian syndrome (PCOS), cardiovascular disease, or eruptive xanthomas. In some instances, the subject has one or more of an autoimmune disease. In some instances, the autoimmune disease is dermatomyositis, polymyositis Sjogren's syndrome, rheumatoid arthritis, scleroderma, systemic sclerosis, and systemic lupus erythematosus. In some instances, the immunosuppressive therapy comprises at least one of an immunosuppressant drug, intravenous immunoglobulin administration, plasma exchange plasmapheresis, immunoadsorption, or administration of the antigenic PLIN1 polypeptide or immunogenic fragments thereof.

### BRIEF DESCRIPTION OF THE DRAWINGS

FIG. 1 shows that autoantibodies to PLIN1 are present in sera from a patient with autoimmune AGL following cancer immunotherapy, according to aspects of this disclosure. Radioligand binding assays screening for PLIN1 antibodies in sera from all time points from the patient are plotted along with checkpoint-treated control patients without lipodystrophy (n=7). Dotted line indicates mean+3 std. dev. of healthy controls (n=11). Data point labels: Pre: patient sample before initiation of checkpoint inhibitor therapy (CPI). 34 courses of CPI (nivolumab) were performed. CPI was discontinued after 16 months due to progressive weight loss and elevated liver function tests (LFTs). 1: 8 weeks after CPI discontinuation; 2: 9 weeks after CPI discontinuation; 3: 5 months after CPI discontinuation; 4: 19 months after CPI discontinuation.

### DETAILED DESCRIPTION

As used in the disclosure and the appended claims, the singular forms “a,” “an,” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations, and methodologies which are described in the publication and which might be used in connection with the presently described invention.

Lipodystrophy traditionally has been classified as congenital or acquired. The exact cause for acquired lipodystrophy is unknown. In some cases, lipodystrophy is associated with panniculitis. This type may manifest with subcutaneous inflammatory nodules (panniculitis), which heal by localized loss of fat and eventually results in complete loss of subcutaneous fat. In other instances, lipodystrophy can develop as a side effect of HIV medication. HIV-associated lipodystrophy is commonly observed in HIV patients treated with anti-retroviral medicines. However, in a large fraction of patients, the underlying mechanism of fat loss is not clear. Although the pathogenesis of both, acquired generalized lipodystrophy (AGL, also known as Lawrence Syndrome) and acquired partial lipodystrophy (APL, also known as Barraquer-Simons Syndrome) is unknown, it is hypothesized to be linked to autoimmune destruction of adipocytes. For example, patients with AGL often present with associated autoimmune diseases, such as dermatomyositis, Sjogren's syndrome, rheumatoid arthritis, scleroderma, systemic sclerosis, and systemic lupus erythematosus. Similarly, some patients with APL have a coinciding autoimmune condition, such as lupus erythematosus, dermatomyositis, and/or polymyositis. Thus, as used herein, the term “autoimmune-related lipodystrophy” refers to lipodystrophy conditions that are caused by autoimmunity and does not include lipodystrophies that are associated with HIV treatment or panniculitis.

Recently, lipodystrophy has been described in patients that underwent immune checkpoint blockade therapy as described by Gnanendran et al. (2020), Melanoma Res. 30(6):599-602. Immune checkpoint blockade therapy, or immune checkpoint inhibitor (ICI) therapy (also referred to as immune checkpoint blockade (ICB) therapy), refers to a cancer immunotherapy that elicits or amplifies an immune response and involves the administration of immune checkpoint inhibitors. Immune checkpoint inhibitors are molecules that totally or partially reduce, inhibit, interfere with or modulate one or more immune checkpoint proteins. Checkpoint proteins share the common function of providing inhibitory signals that suppress immune response. That is, checkpoint proteins help keep immune responses from being too strong. In particular, checkpoint proteins regulate T-cell activation or function and are responsible for co-stimulatory or inhibitory interactions of T-cell responses. Thus, immune checkpoint proteins regulate and maintain peripheral tolerance to self-antigens, so that the immune system does not attack a person's own normal cells. They also regulate the duration and amplitude of physiological immune responses. Numerous checkpoint proteins are known, such as CTLA-4 and its ligands CD80 and CD86; and PD-1 with its ligands PD-L1 and PD-L2 as discussed, e.g., in Pardoll, Nature Reviews Cancer 12: 252-264 (2012). Other immune checkpoint targets include B7, CD137, T-cell immunoglobulin and mucin domain-3 (TIM-3), lymphocyte activation gene-3 (LAG-3), V-domain immunoglobulin suppressor of T cell activation (VISTA), and T cell immunoreceptor with immunoglobulin and immunoreceptor tyrosine-based inhibitory motif domains (TIGIT).

Inhibition of one or more immune checkpoint proteins can block or otherwise neutralize inhibitory signaling, resulting in an upregulated an immune response. A stronger immune response can more efficaciously treat certain disease, such as cancer. While ICI therapies have led to improved outcomes for many cancer patients, they have also introduced a risk for immune-related adverse events (IRAEs). The therapeutic benefit of inhibiting immune checkpoint proteins is thought to result from the amplification of suppressed anti-tumor immune responses that are blocked by tumor-specific manipulations of the immune system. However, these regulatory pathways are also involved in the regulation of autoimmune immune responses, i.e. whether the immune system of an organism responds against its own healthy cells and tissues (autoimmunity). The onset and outcome of IRAEs seems to vary according to the organs involved and, although most occur within the first 3 months of treatment, there are some specific toxicities reported months after the end of therapy.

The present disclosure describes a specific Immunoglobulin G (IgG) autoantibody marker that has been identified for the early detection of autoimmune-related lipodystrophy, or for assessing the risk of developing autoimmune-related lipodystrophy, in patients during or following ICI therapy. The target of this novel autoantibody marker has been identified as Perilipin 1 (PLIN1). Accordingly, this disclosure describes methods and compositions for immunohistochemically detecting the presence of PLIN1 autoantibodies in a biological sample. For example, this disclosure provides for methods of identifying a subject that is at risk of developing lipodystrophy following or in the course of ICI therapy. Provided are also for methods of detecting PLIN1 autoantibodies in a subject that presents with signs and symptoms of lipodystrophy and has been treated or is being treated with ICI therapy. In some embodiments, provided are methods of determining if a subject that has been treated or is being treated with ICI therapy and presents with signs and symptoms of lipodystrophy is serologically positive for a PLIN1 autoantibody. Also provided are methods of treating a subject that has received or is receiving ICI therapy and is serologically positive for one or more PLIN1 autoantibodies.

As summarized in the examples of this disclosure, PLIN1 autoantibodies are not found in healthy subjects or in subjects with cancer that have been treated with ICI therapy but do not develop a lipodystrophy condition. In some instances, the methods provided herein are useful in monitoring patients who are receiving or have previously received ICI therapy to detect early evidence of lipodystrophy or development of lipodystrophy, or assess the risk of developing lipodystrophy. In some instances, the methods may identify a subject that does not have particular symptoms associated with lipodystrophy but who is as at risk of developing such condition following or in the course of receiving ICI therapy. Such patients can then be monitored for development of such symptoms so that appropriate treatment can be administered prophylactically and/or promptly. In some cases, ICI therapy in such patients may be altered or terminated in order to prevent the development or progression of lipodystrophy and/or associated conditions.

In some instances, the methods provided herein are useful for the serological evaluation of subjects who undergo ICI therapy and present with lipodystrophy of undetermined etiology, to assist in clinical diagnosis and ultimately appropriate therapeutic intervention. In some instances, the provided methods are useful for investigating lipodystrophy associated symptoms that appear in the course of, or after, a subject receiving ICI therapy and that are not explainable by cancer and/or metastasis. In some instances, the provided methods are useful for confirming the presence of lipodystrophy in a subject that is being treated or has been treated with ICI therapy and is suspected to have lipodystrophy. Thus, the methods provided herein are useful for the evaluation of subjects for the presence of PLIN1 autoantibodies where such patients present with one or more clinical symptoms associated with lipodystrophy following or during ICI therapy, so as to assist in clinical diagnosis and appropriate therapeutic intervention.

Perilipins (PLINs) are evolutionarily conserved proteins that share sequence homology and the ability to bind lipid droplets, but differ in their sizes, tissue expression profiles, transcriptional regulation and affinities for lipid droplets. Depending on their expression patterns, PLINs can be classified into two groups: PLINs expressed in a tissue-restricted manner (PLIN1, PLIN4 and PLIN5) and those with ubiquitous expression (PLIN2 and PLIN3). Besides, some PLINs are constitutively bound to lipid droplets (PLIN1 and PLIN2) while others exhibit exchangeable lipid droplet binding (PLIN3, PLIN4 and PLIN5). PLIN1 is the best characterized member of the perilipin family, with six phosphorylation sites (Ser-81, Ser-223, Ser-277, Ser-434, Ser-492, and Ser-517). The human PLIN1 is 522 amino acids in length. See UniProt Database Entry UniProt/UniProtKB Database Entry 060240.

PLIN1 is expressed in adipocytes where it remains bound to the cytosolic lipid droplet and participates in the regulation of triacylglycerol storage and breakdown. Under basal conditions, PLIN1 restricts the access of cytosolic lipases to the neutral lipid core, thus promoting triacylglycerol storage. The two major lipolytic enzymes are adipose triacylglycerol lipase (ATGL) and hormone-sensitive lipase (HSL), both of which are cytosolic. Under basal (e.g., fed or insulin-stimulated) conditions, PLIN1 is unphosphorylated and, via its C-terminal portion, hijacks the comparative gene identification-58 (CGI-58) protein, an activator of ATGL, to suppress lipolysis. PLIN1 is available for rapid phosphorylation upon b-adrenergic stimulation. C-terminal phosphorylation of PLIN1 disrupts the interaction with CGI-58, allowing it to recruit, mobilize, and activate ATGL, which catalyzes the initial step of lipolysis. N-terminal phosphorylation of PLIN1 is essential for HSL recruitment on the lipid droplet surface. HSL is the major enzyme responsible of diglyceride hydrolysis. This is evidenced by the phenotype of PLIN1 null mice, which have a reduction of adipose triacylglycerol storage compared with wild-type animals and are resistant to diet-induced obesity. Adipocytes isolated from PLIN1 null mice have a higher rate of basal lipolysis, which is consistent with the role for PLIN1 in protecting the core of the lipid droplet from the action of lipases.

A. Methods of Detection and Compositions

In one aspect, a PLIN1 antigenic polypeptide or fragment or variant thereof can be used in various immunological techniques to detect PLIN1-specific autoantibodies. The entire PLIN1 protein can used in the provided methods, a fragment of the PLIN1 protein may be used, a variant of the PLIN1 protein may be used, or a combination of two or more of the full length polypeptide, a fragment, or a variant thereof as described in this disclosure may be used. For example, PLIN1 antigenic polypeptides can be used in an immunoassay to detect PLIN1 autoantibodies in a biological sample. PLIN1 antigenic polypeptides used in an immunoassay can be in a cell lysate (such as, for example, a whole cell lysate or a cell fraction), or purified PLIN1 polypeptides or fragments thereof can be used provided at least one antigenic site recognized PLIN1-specific autoantibodies (such as PLIN1 autoantibodies) remains available for binding.

In another aspect, provided are methods of identifying a subject that has been previously treated or is being treated with immune checkpoint blockade therapy and is at risk of developing lipodystrophy, comprising the steps of contacting the biological sample with a PLIN1 antigenic polypeptide or fragment or variant thereof and detecting the presence of binding of the PLIN1 antigenic polypeptide or fragment thereof to PLIN1 autoantibodies in the biological sample.

In one aspect, provided are methods of detecting the presence of a PLIN1 autoantibody in a biological sample from a subject that has been previously treated or is being treated with immune checkpoint blockade therapy, comprising the steps of contacting the biological sample with a PLIN1 antigenic polypeptide or fragment or variant thereof and detecting the presence of binding of the PLIN1 antigenic polypeptide or fragment thereof to PLIN1 autoantibodies in the biological sample.

The term “autoantibody”, as used herein, refers to an antibody that specifically binds to an endogenous molecule in a subject that produces said autoantibody. The level of such antibody is typically elevated compared the average of any other antibodies binding specifically to such an endogenous molecule. The endogenous molecule may be an autoantigen. An autoantigen is defined as protein or protein complex (and sometimes DNA or RNA) that is recognized by the immune system (e.g., through autoantibodies) of a subject suffering from a specific autoimmune disease. These antigens should not be, under normal conditions, the target of the immune system, but T cells instead attack cells expressing the autoantigens.

A “biological sample,” as used herein, generally refers to a bodily tissue or fluid obtained from a human, preferably a mammalian subject. Exemplary subjects include, but are not limited to humans, non-human primates such as monkeys, dogs, cats, mice, rats, cows, horses, camels, goats, and sheep. In some embodiments, the subject is a human. Non-limiting examples of biological samples include blood, blood fractions or blood products (e.g., serum, plasma, platelets, red blood cells, peripheral blood mononuclear cells and the like), sputum or saliva, stool, urine, other biological fluids (e.g., lymph, prostatic fluid, gastric fluid, intestinal fluid, renal fluid, lung fluid, cerebrospinal fluid, and the like). Additionally, solid tissues, for example, tissue biopsies (e.g., subcutaneous fat tissue) may be used. A biological sample may be processed prior to use in a detection assay including dilution, addition of buffer or preservative, concentration, purification, or partial purification.

In the context of this disclosure, the subject has cancer or has been treated for cancer and no longer has cancer. In some instances, the subject has cancer. In some instances, the subject has previously had cancer. In some instances, the subject is undergoing treatment for cancer when diagnosed with lipodystrophy or when one or more signs and/or symptoms of lipodystrophy appear in the subject. In some instances, the subject does not have cancer (i.e. is in remission) when diagnosed with lipodystrophy or when signs and symptoms of lipodystrophy appear in the subject.

The term “cancer” refers to a disease characterized by the uncontrolled growth of aberrant cells. The term includes all known cancers and neoplastic conditions, whether characterized as malignant, soft tissue, or solid, and cancers of all stages and grades including pre- and post-metastatic cancers, as well as recurrent cancer. Examples of different types of cancer include, but are not limited to, digestive and gastrointestinal cancers such as gastric cancer (e.g., stomach cancer), colorectal cancer, gastrointestinal stromal tumors, gastrointestinal carcinoid tumors, colon cancer, rectal cancer, anal cancer, bile duct cancer, small intestine cancer, esophageal cancer, breast cancer, lung cancer (e.g., non-small cell lung cancer), gallbladder cancer, liver cancer, pancreatic cancer, appendix cancer, prostate cancer, ovarian cancer, cervical cancer, uterine cancer, renal cancer, cancer of the central nervous system, skin cancer (e.g., melanoma), lymphomas, gliomas, choriocarcinomas, head and neck cancers, osteogenic sarcomas, and blood cancers. In some embodiments, the cancer is melanoma. In some cases, the cancer may be recurrent, metastatic melanoma.

In general, the presence of PLIN1 autoantibodies is measured in a subject that has previously been treated or is being treated with ICI therapy. Immune checkpoint inhibitors can include antibodies and antibody fragments, nanobodies, diabodies, soluble binding partners of checkpoint molecules, small molecule therapeutics, or peptide antagonists, as well as RNA interference, antisense, and nucleic acid aptamers that can downregulate the expression and/or activity of immune checkpoint nucleic acids, or fragments thereof. Exemplary immune checkpoint inhibitors include CTLA-4 blocking antibodies, anti-PD-1 antibodies, anti-PD-L1 antibodies, and/or anti-PD-L2 antibodies, either alone or in combination. Numerous immune checkpoint inhibitors are known and publicly available including, for example, Keytruda® (pembrolizumab; anti-PD-1 antibody), Opdivo® (nivolumab; anti-PD-1 antibody), Tecentriq® (atezolizumab; anti-PD-L1 antibody), durvalumab (anti-PD-L1 antibody), Yervoy® (ipilimumab; anti-CTLA-4 antibody) and the like. In specific embodiments, the checkpoint blockade therapy may include an anti-PD-1 antibody. In one embodiment, the immune checkpoint inhibitor is nivolumab.

In some embodiments, the subject has signs and symptoms associated with lipodystrophy. In some embodiments, the subject has lipodystrophy or is suspected to have lipodystrophy. In some embodiments, the subject is diagnosed with lipodystrophy. The lipodystrophy may be AGL or APL. In some embodiments, the subject has one or more of insulin resistance, weight loss, low serum leptin, hyperphagia, polydipsia, dyslipidemia, acute pancreatitis, hepatic cirrhosis, proteinuria and renal failure, or polyuria. In some embodiments, the subject presents with one or more of an autoimmune disease. The autoimmune disease may be dermatomyositis, polymyositis Sjogren's syndrome, rheumatoid arthritis, psoriatic arthritis, ankylosing spondylitis, scleroderma, systemic sclerosis, Graves' disease, Hashimoto's thyroiditis, or systemic lupus erythematosus. In some embodiments, the subject has an autoimmune associated condition such as one or more of type II diabetes, hypertriglyceridemia, hepatic steatosis, steatohepatitis, acanthosis nigricans, polycystic ovarian syndrome (PCOS), cardiovascular disease, or eruptive xanthomas.

In some embodiments, the subject has metabolic abnormalities. As used herein, the term “metabolic abnormality” means any abnormal condition in an individual caused by a defect in one or more metabolic pathways. Metabolism is the physiological and biochemical process by which food is converted in the body to forms that provide energy for bodily activities, e.g., the creation of specific molecules. Such molecules include hormones, neurotransmitters, proteins such as enzymes, and membrane constituents. Metabolism also includes degradation processes that enable cells to excrete waste products. Exemplary metabolic processes include processes for absorption and modification of vitamins and minerals, for degrading molecules to provide energy or to be excreted, or processes for making acetyl-coenzyme A, nonessential amino acids, cholesterol, long-chain fatty acids, prostaglandins, complex lipids and proteins; and for neutralizing toxins. Metabolic abnormalities include, for example, abnormalities in glucose metabolism, lipid metabolism, and hormone metabolism.

In some embodiments, the subject has insulin resistance. Tests that can determine whether a subject has insulin resistance are known in the art and include those that measure fasting insulin and glucose levels in a sample to calculate insulin resistance. Generally, a fasting (or three hours after the last meals) serum insulin level greater than 25 mU/L or 174 pmol/L indicates insulin resistance. Tests for measuring insulin and/or glucose levels are known in the art and include, for example, glucose tolerance testing, glycated hemoglobin (HbA1c or Ac1) testing, hyperinsulinemic euglycemic clamp, modified insulin suppression test, Homeostatic Model Assessment (HOMA), Quantitative insulin sensitivity check index (QUICKI). See e.g., Muniyappa et al. (2008), “Current approaches for assessing insulin sensitivity and resistance in vivo: advantages, limitations, and appropriate usage,” American Journal of Physiology Endocrinology and Metabolism. 294 (1): E15-26; Borai et al. (2011), “Selection of the appropriate method for the assessment of insulin resistance. BMC Med. Res. Methodol. 11:158.

In some embodiments, the subject has lipid abnormalities, such as, for example, dyslipidemia. Dyslipidemia is characterized by elevated triglycerides and low high-density lipoprotein (HDL) cholesterol. Several diagnostic tests may be used to diagnose lipid abnormalities and are known in the art. Tests include, for example, a cholesterol test (lipid panel), which is a commonly used blood test that measures levels of cholesterol, triglycerides, high-density lipoprotein (HDL) and low-density lipoprotein (LDL), advanced lipid profile testing, which is a more sophisticated test that goes beyond standard screening to further identify the various components of cholesterol, and lipoprotein(a) test, which is used to measure the amount of LDL or low-density lipoprotein in the blood. Lipoprotein(a) is a lesser known type of LDL. See e.g., Jialal (1996), “A practical approach to the laboratory diagnosis of dyslipidemia,” 106(1): 128-38.

In some cases, the subject has steatosis. Steatosis refers to abnormal retention of fat (lipids) within a cell or organ. Steatosis most often affects the liver where the condition is commonly referred to as fatty liver disease. Steatosis may be diagnosed, for example, by performing histological examination of biopsied liver tissue. In some cases, steatosis may be diagnosed using medical imaging, such as X-ray computed tomography (where increased fat components decrease the liver tissue tensity, making the image less bright), magnetic resonance imaging that can be used to determine the percent fat fraction in the liver, or abdominal ultrasonography. See e.g, Helms et al. (2007), “Fundamentals of diagnostic radiology,” Phila: Lippincott, Williams & Wilkins. ISBN 978-0-7817-6135-2; Reeder et al. (2011). “Quantitative Assessment of Liver Fat with Magnetic Resonance Imaging and Spectroscopy,” J Magn Reson Imaging. 34 (4): 729-749.

In some embodiments, the subject has decreased levels of leptin. Various diagnostic tests may be used to determine if there is a decrease in leptin. Leptin is a hormone that helps regulate appetite by signaling hunger satisfaction (satiety). This test measures the amount of leptin in the blood to detect a deficiency that may be contributing to obesity. Leptin is produced primarily by fat cells (adipocytes). Insufficient leptin can cause persistent hunger as the body attempts to protect itself from perceived underfeeding (starvation). For men and women with a body mass index (BMI) of 22, leptin values are normally 0.7 and 5.3 ng/mL and 3.3 and 18.3 ng/mL, respectively. Blood leptin levels can be measured by routine testing such as enzyme immunoassays and electrochemiluminescence.

Generally, the biological sample is assessed for the presence of PLIN1 autoantibodies by contacting the biological sample with a PLIN1 antigenic polypeptide or fragment or variant thereof. In some embodiments, the PLIN1 polypeptide or fragment thereof is present in a solid tissue such as a tissue section. For example, a tissue sample comprising PLIN1 polypeptides or fragments may be used, which may be in the form of a tissue section fixed on a carrier, for example a glass slide for microscopic analysis. For example, the solid tissue can be subcutaneous fat tissue or tissue biopsies containing other fatty tissue. In some embodiments, the PLIN1 polypeptide or fragment thereof is present in a sample from a mammal. For example, mouse tissue is routinely used in immunohistochemistry, as described in the Examples below, but tissue from other rodents (e.g., rats) or other mammals (e.g., rabbits, non-human primates, or humans) can also be used in the present methods. In some embodiments, the tissue may be mouse enteric tissue or adipose tissue. Tissue sections used in immunohistochemistry are well known in the art and are commercially available from a number of companies (e.g., Asterand, Inc. (Detroit, Michigan); Euroimmun (Morris Plains, New Jersey); and Imgenex (San Diego, California)). In other embodiments, the PLIN1 polypeptide or fragment thereof is in a cell lysate, blood, serum, cerebrospinal fluid (CSF), or urine.

In some embodiments, a liquid sample comprising PLIN1 autoantibodies from a subject may be used to practice the methods provided herein. Exemplary liquid samples include cell lysate, blood, serum, cerebrospinal fluid (CSF), and urine. A step of contacting a liquid sample comprising PLIN1 autoantibodies with a PLIN1 antigenic polypeptide or fragment or variant thereof may be carried out by incubating an immobilized form of said polypeptide in the presence of the liquid sample under conditions that are compatible with the formation of a complex comprising said polypeptides and said PLIN1 autoantibodies. Optionally, one or more washing steps may be contemplated.

In some embodiments, the PLIN1 polypeptide or fragment thereof is an isolated, purified PLIN1 polypeptide or fragment thereof as discussed below. In some embodiments, the PLIN1 polypeptide or fragment thereof is in a phage display or eukaryotic cell display library. In some embodiments, the PLIN1 polypeptide or fragments thereof is heterologously-expressed on the surface of a cell.

In some embodiments, the biological sample is contacted with a PLIN1 polypeptide or fragment thereof and a secondary antibody. As is well known in the art, the secondary antibody is an antibody raised against the IgG of the animal species in which the primary antibody originated. Secondary antibodies bind to the primary antibody to assist in detection, sorting and purification of target antigens to which a specific primary antibody is first bound. The secondary antibody must have specificity both for the antibody species as well as the isotype of the primary antibody being used. If a PLIN1 autoantibody is present in the biological sample, under appropriate conditions, a complex is formed between the PLIN1 polypeptide or fragment thereof, the PLIN1 autoantibody in the biological sample, and the secondary antibody.

A complex comprising the PLIN1 autoantibodies and PLIN1 polypeptides or fragments may be detected using a variety of methods known to the person skilled in the art, for example immunofluorescence microscopy or spectroscopy, luminescence, NMR spectroscopy, immunodiffusion, radioactivity, chemical crosslinking, surface plasmon resonance, native gel electrophoresis, or enzymatic activity. Depending on the nature of the sample, either or both immunoassays and immunocytochemical staining techniques may be used. Enzyme-linked immunosorbent assays (ELISA), Western blot, and radioimmunoassays are methods used in the art, and can be used as described herein to detect the presence of PLIN1 autoantibodies in a biological sample. While some of these methods allow for the direct detection of the complex, in some embodiments, the second antibody is labeled such that the complex may be detected specifically owing to intrinsic properties of the label such as, for example, fluorescence, radioactivity, enzymatic activity, visibility in NMR, or MRI spectra or the like. In some embodiments, the detection method may include any of Western blot, dot blot, protein microarray, ELISA, line blot radioimmune assay, immunoprecipitation, indirect immunofluorescence microscopy, radioimmunoassay, radioimmunodiffusion, ouchterlony immunodiffusion, rocket immunoelectrophoresis, immunohistostaining, complement fixation assay, FACS, and protein chip, but is not limited thereto. Methods and compositions are described herein that can be used for detecting, by immunohistochemistry, the presence of PLIN1 autoantibodies in a biological sample. Immunohistochemical methods are well known in the art, and non-limiting exemplary methods are described in U.S. Pat. Nos. 5,073,504; 5,225,325; and 6,855,552. See also Dabbs, Diagnostic Immunohistochemistry, 2^{nd} Ed., 2006, Churchill Livingstone; and Chu & Weiss, Modern Immunohistochemistry, 2009, Cambridge University Press. It would be understood by those skilled in the art that immunohistochemistry routinely includes steps that are not necessarily discussed herein in detail such as washing the tissue samples to remove unbound secondary antibodies and the parallel staining experiments with proper controls. Exemplary detection methods are described in the Examples of this disclosure, including radiolabeled ligand binding assays, immunofluorescence, and cell-based expression assays. While particular protocols are described below, variations of these assays are routine and known in the art.

In some instances, the secondary antibody is conjugated to a detectable label. Detectable labels are well known in the art and include, without limitation, a fluorescent label, an enzymatic label, a radioactive label, a luminescent label, or an affinity tag such as biotin or streptavidin. Exemplary fluorescent dyes include water-soluble rhodamine dyes, fluoresceins, 2′,7′-dichlorofluoresceins, fluorescein isothiocyanate (FITC), DyLight™ 488, phycoerythrin (PE), propidium iodide (PI), PerCP, PE-Alexa Fluor® 700, Cy5, allophycocyanin, Cy7, benzoxanthene dyes, and energy transfer dyes, as disclosed in the following references: Handbook of Molecular Probes and Research Reagents, 8^{th} ed. (2002), Molecular Probes, Eugene, OR; U.S. Pat. Nos. 6,191,278, 6,372,907, 6,096,723, 5,945,526, 4,997,928, and 4,318,846; and Lee et al., 1997, Nucleic Acids Research 25:2816-2822. Exemplary enzymatic labels include but are not limited to alkaline phosphatase (AP) and horseradish peroxidase (HP)). Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Suitable radioactive labels include, for example, ³²P, ³³P, ¹⁴C, ¹²⁵I, ¹³¹I, ³⁵S, and ³H. In some instances, the detectable label can be a heterologous polypeptide such as an antigenic tag such as, for example, FLAG, polyhistidine, hemagglutinin (HA), glutathione-S-transferase (GST), or maltose-binding protein (MBP)) for use in purifying the PLIN1 polypeptide or antigenic fragments or variants thereof. In some instances, the detectable label can be a heterologous polypeptide that is useful as diagnostic or detectable marker such as, for example, luciferase, a fluorescent protein (such as a green fluorescent protein (GFP)), or chloramphenicol acetyl transferase (CAT). Another labeling technique which may result in greater sensitivity is the coupling the antibodies to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten antibodies.

In some embodiments, the method comprises contacting a PLIN1 antigenic polypeptide or fragment or variant thereof with a biological sample from a subject and a secondary antibody having a suitable label thereon under conditions in which a complex is formed between the PLIN1 polypeptide or antigenic fragment or variant thereof, a corresponding PLIN1 autoantibody in the biological sample, if present, and the secondary antibody; and detecting the complex formed, if formed, by detecting the label of the secondary antibody, wherein the presence of the secondary antibody is indicative of the presence of a PLIN1 autoantibody in the biological sample, and wherein the absence of the secondary antibody is indicative of the absence of a PLIN1 autoantibody in the biological sample. In some instances, the secondary antibody is detectably-labeled. Immobilization of the PLIN1 polypeptide or antigenic fragment or variant thereof on a solid carrier can facilitate the method of PLIN1 autoantibody detection as discussed below. In some instances, the method comprises contacting a PLIN1 polypeptide or antigenic fragment or variant thereof having a suitable label thereon with a biological sample from a subject, and immunoprecipitating any complex formed between the PLIN1 polypeptide or antigenic fragment or variant thereof and a corresponding PLIN1 autoantibody in the biological sample, and monitoring for said label on any of said complexes, wherein the presence of said label is indicative of the presence of a PLIN1 autoantibody in the biological sample and the absence of said label is indicative of the absence of a PLIN1 autoantibody in the biological sample. In some instances, the method comprises a combination of immunoprecipitation and Western blot analysis to detect the presence of a PLIN1 autoantibody in a biological sample from a subject. For example, the method may comprise contacting a PLIN1 antigenic polypeptide or fragment or variant thereof with a biological sample from a subject under conditions in which a complex is formed between the PLIN1 polypeptide or antigenic fragment or variant thereof and a corresponding PLIN1 autoantibody in the biological sample, if present; immunoprecipitating any complex formed between the PLIN1 polypeptide or antigenic fragment or variant thereof and a corresponding PLIN1 autoantibody in the biological sample to produce an immunoprecipitate comprising any such complex formed; separating components of the immunoprecipitate from each other (e.g., by electrophoresis), said components comprising the PLIN1 polypeptide or antigenic fragment or variant thereof and a corresponding PLIN1 autoantibody in the biological sample, if present; and contacting the components of the immunoprecipitate with a secondary antibody having a suitable label thereon that specifically binds to a constant region of the PLIN1 autoantibody, if present; and detecting the complex formed, if formed, by detecting the label of the secondary antibody, wherein the presence of the secondary antibody is indicative of the presence of a PLIN1 autoantibody in the biological sample, and wherein the absence of the secondary antibody is indicative of the absence of a PLIN1 autoantibody in the biological sample. For example, immunoprecipitation assay may be performed to detect the presence of PLIN1 autoantibodies in a subject by contacting recombinant PLIN1 protein with a biological sample from the subject, such as serum. Exemplary labels include any of the detectable labels described in this disclosure including, for example, fluorescent dyes and radioactive labels.

In some embodiments, the PLIN1 polypeptide or antigenic fragment or variant thereof is heterologously-expressed on the surface of a cell. For example, a vector comprising the coding sequence of the PLIN1 polypeptide or antigenic fragment or variant thereof operably linked to a promoter can be introduced into a cell. The vector may comprise elements that cause the PLIN1 polypeptide or fragment thereof to be expressed on the surface of the cell. For example, the PLIN1 polypeptide fragments and variants thereof may be expressed as fusion proteins with a membrane protein on the surface of the cell. In some instances, the cell is a bacteria cell or a eukaryotic cell. For example, the eukaryotic cell may be a yeast cell or a mammalian cell such as a human cell. Methods of transfection and transduction of cells to introduce recombinant nucleic acids are well known in the art. For example, a 293T cell-based expression assay can be used to a PLIN1 commercial antibody, as well as PLIN1 autoantibodies in a biological sample from a subject, such as serum.

In some embodiments, an isolated, purified PLIN1 polypeptide or antigenic fragment or variant thereof may be used in the provided methods. Protein expression and purification methods are well known in the art. In some embodiments, the PLIN1 polypeptide has the sequence represented by UniProt/UniProtKB Database Entry 060240 (SEQ ID NO:1), which refers to the sequence deposited in the UniProtKB and NCBI databases, more specifically the version publicly available on Dec. 16, 2020. However, the teachings of the present invention may not only be carried out using polypeptides, in particular a polypeptide comprising the full-length sequence of PLIN1, having the exact amino acid residue sequences referred to in this application explicitly, for example by name, sequence or accession number, or implicitly, but also using fragments or variants of such polypeptides. Thus, modified PLIN1 polypeptides and antigenic fragments or variants thereof are also contemplated, such as those in which one or more amino acid residues are substituted or modified (such as with glutaraldehyde).

An “isolated” or “purified” polypeptide, or portion thereof, is substantially or essentially free from components that normally accompany or interact with the polypeptide or protein as found in its naturally occurring environment. Thus, an isolated or purified polypeptide or protein is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, or 1% (by dry weight) of contaminating protein. When the PLIN1 polypeptide or antigenic portion thereof is recombinantly produced, optimally culture medium represents less than about 30%, 20%, 10%, 5%, or 1% (by dry weight) of chemical precursors or non-protein-of-interest chemicals.

The term “fragment” with regard to PLIN1 refers to an amino acid residue sequence of a portion of the full-length protein, encompassing, for example, an amino acid residue sequence that is truncated at one or both termini by one or more amino acids. The PLIN1 polypeptide fragment retains its antigenicity such that it is bound specifically under appropriate binding conditions by a PLIN1 autoantibody that would bind specifically to the corresponding full-length PLIN1 protein under appropriate binding conditions. An antigenic portion of the PLIN1 protein can be a polypeptide that is, for example, 10, 25, 50, 100, 150, 200, 250 or more amino acid residues in length of the full length PLIN1 protein. Alternatively or in addition, such peptide sequence may comprise one or more internal deletions of one or more amino acid residues. Thereby the residual length of the fragment equals or exceeds the length of one or more continuous or conformational epitopes, e.g., 6, 7, 8, 9, 10, 11, 12, 13, 14, 15, 16, 17, 18, 19, 20, 21, 22, 23, 24, 25, 26, 27, 28, 29, 30, 21, 32, 33, 34, 35, 36, 37, 38, 39, 40, 41, 42, 43, 44, 45, 46, 47, 48, 49, 50, 55, 60, 65, 70, 75, 80, 85, 90, 95, 100 or more amino acid residues. In some embodiments, a fragment comprises at least 6 contiguous amino acid residues of SEQ ID NO:1. In some embodiments, a fragment comprises at least 8 contiguous amino acid residues of SEQ ID NO:1. In some embodiments, a fragment comprises at least 12 contiguous amino acid residues of SEQ ID NO:1. In some embodiments, a fragment comprises 8-12 contiguous amino acid residues of SEQ ID NO:1. In some embodiments, a fragment comprises 30-60 contiguous amino acid residues of SEQ ID NO:1. In some embodiments, a fragment comprises 47 contiguous amino acid residues of SEQ ID NO:1. In some instances, a plurality of fragments is provided, each fragment comprising 49 contiguous amino acid residues of SEQ ID NO:1 such as, for example, the fragments identified in Table 1. For example, a first fragment may comprise amino acid residues 1-49 of SEQ ID NO:1, a second fragment comprising amino acid residues 25-73, a third fragment comprising amino acid residues 49-97 of SEQ ID NO:1, and so on with each additional fragment having a first amino acid residue 24 amino acids downstream in the amino acid sequence of SEQ ID NO:1 relative to the first amino acid residue of the prior fragment. In some embodiments, the antigenic PLIN1 polypeptide fragments include those comprising the sequence of any of SEQ ID NOs: 2-23 or more than one thereof. In some embodiments, the antigenic PLIN1 polypeptide fragments include those comprising the sequence of any of SEQ ID NOs: 2-6 or more than one thereof.

The person of skill in the art is familiar with guidelines used to design peptides having sufficient immunogenicity such as, for example, those described in Jackson, D. C., et al., Vaccine 18(3-4): 355-361 (1999) and Black, M., et al., Expert Rev. Vaccines, 9(2): 157-173 (2010). Briefly, it is desirable that the peptide meets as many as possible of the following requirements: (a) it has a high degree of hydrophilicity, (b) it comprises one or more residues selected from the group comprising aspartate, proline, tyrosine, and phenylalanine, (c) is has, for higher specificity, no or little homology with other known peptides or polypeptides, (d) it is sufficiently soluble, and (e) it comprises no glycosylation or phosphorylation sites unless required for specific reasons. Alternatively, bioinformatics approaches may be followed such as, for example, those described by Moreau, V., et al., BMC Bioinformatics 2008, 9:71 (2008). Such biologically active portions can be prepared by recombinant techniques and evaluated for pesticidal activity.

The term “variant” of PLIN1, or fragments thereof, refers to a polypeptide comprising an amino acid residue sequence that is at least 70, 75, 80, 85, 90, 92, 94, 95, 96, 97, 98 or 99% identical to the normal sequence of the PLIN1 protein, or a fragment thereof. Within the context of this disclosure, a variant of the PLIN1 protein, or a fragment thereof, retains its antigenicity such that it is bound specifically under appropriate conditions by a PLIN1 autoantibody that would specifically bind to the corresponding full length PLIN1 polypeptide under appropriate conditions. In some instances, variants are modified at amino acid residues other than those essential for the biological activity, for example the ability of an antigen to bind specifically to a PLIN1-specific antibody, such as a PLIN1 autoantibody. In some instances, one or more such essential amino acid residues may optionally be replaced in a conservative manner or additional amino acid residues may be inserted such that the biological activity (i.e. antigenicity) of the variant polypeptide is preserved. Methods of alignment of sequences for comparison are well known in the art, e.g., visual alignment or using publicly available software using known algorithms to achieve maximal alignment. The state of the art comprises various methods that may be used to align two given nucleic acid or amino acid sequences and to calculate the degree of identity, see for example Arthur Lesk (2008), Introduction to bioinformatics, Oxford University Press, 2008, 3rd edition. In a preferred embodiment, the ClustalW software (Larkin, M. A., Blackshields, G., Brown, N. P., Chenna, R., McGettigan, P. A., McWilliam, H., Valentin, F., Wallace, I. M., Wilm, A., Lopez, R., Thompson, J. D., Gibson, T. J., Higgins, D. G. (2007). Clustal W and Clustal X version 2.0. Bioinformatics, 23, 2947-2948) is used. Alternatively, alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)). Other publicly available software useful for alignment analysis include BLAST programs, ALIGN, ALIGN-2 (Genentech, South San Francisco, Calif.), and Megalign (DNASTAR).

Such variants of PLIN1 and fragments thereof may be prepared, for example, by introducing deletions, insertions or substitutions in nucleic acid sequences encoding them, or by chemical synthesis or modification. Moreover, variants of PLIN1 and fragments thereof may also be generated by fusion with other known polypeptides or variants thereof and encompass active portions or domains, preferably having a sequence identity of at least 70, 75, 80, 85, 90, 92, 94, 95, 96, 97, 98 or 99% when aligned with the active portion of the reference sequence, wherein the term “active portion”, as used herein, refers to an amino acid sequence, which is less than the full length amino acid sequence or, in the case of a nucleic acid sequence, codes for less than the full length amino acid sequence, respectively, but retains at least some of the biological activity. For example, an active portion an antigenic polypeptide retains the ability to bind to an antibody or autoantibody and, preferably, when administered to mammals, causes an immune response to occur.

The one or more PLIN1 antigenic polypeptides and fragments and variants thereof may be provided in any form and at any degree of purification, from tissues or cells comprising said polypeptides in an endogenous form, such as cells overexpressing the polypeptide and crude or enriched lysates of such cells, to purified and/or isolated polypeptides that are essentially pure. In embodiments, the one or more PLIN1 polypeptides or antigenic fragments or variants thereof have a native configuration, wherein the term “native configuration”, as used herein, refers to a folded polypeptide, such as a folded polypeptide purified from tissues or cells, such as mammalian cells or tissues or from non-recombinant tissues or cells. In another embodiment, the one or more PLIN1 polypeptides or antigenic fragments or variants thereof are recombinant proteins, wherein the term “recombinant”, as used herein, refers to a polypeptide produced using genetic engineering approaches at any stage of the production process, for example by fusing a nucleic acid encoding the polypeptide to a strong promoter for overexpression in cells or tissues or by engineering the sequence of the polypeptide itself. Such techniques are well known in the art.

In some instances, the one or more PLIN1 polypeptides or antigenic fragments or variants thereof can be denatured such as by heating, freezing or ultraviolet ray, or chemical treatments such as a surfactant or a denaturant. For example, such a denatured form may be prepared by treating them with sodium dodecyl sulfate (SDS) or dithiothreitol (DTT). PLIN1 polypeptides or antigenic fragments or variants thereof that are included in a kit or a panel as described herein can be provided within a cell, in a solution in which they are soluble, or the PLIN1 polypeptides or fragments or variants thereof can be provided in a lyophilized form.

In some embodiments, the one or more PLIN1 polypeptides or antigenic fragments or variants thereof can be immobilized on a solid carrier insoluble in an aqueous solution, such as via a covalent bond, electrostatic interactions, encapsulation or entrapment, for example by denaturing a globular polypeptide in a gel, or via hydrophobic interactions such as via one or more covalent bonds. Various suitable carriers, for example paper, metal, silicon or glass surfaces, microfluidic channels, membranes, beads such as magnetic beads, column chromatography media, biochips, polyacrylamide gels and the like have been described in the literature, for example in Kim, D., Herr, A. E. (2013), Protein immobilization techniques for microfluidic assays, Biomicrofluidics 7(4), 041501. This way, the immobilized molecule, together with the insoluble carrier, may be separated from an aqueous solution in a straightforward manner, for example by filtration, centrifugation or decanting. An immobilized molecule may be immobilized in a reversible or irreversible manner. For example, the immobilization is reversible if the molecule interacts with the carrier via ionic interactions that can be masked by addition of a high concentration of salt or if the molecule is bound via a cleavable covalent bond such as a disulfide bridge which may be cleaved by addition of thiol-containing reagents. By contrast, the immobilization is irreversible if the molecule is tethered to the carrier via a covalent bond that cannot be cleaved in aqueous solution, for example a bond formed by reaction of an epoxide group and an amine group as frequently used to couple lysine side chains to affinity columns. The protein may be indirectly immobilized, for example by immobilizing an antibody or other entity having affinity to the molecule, followed by formation of a complex to the effect that the molecule-antibody complex is immobilized. Various ways to immobilize molecules are described in the literature such as, for example, in Kim and Herr (2013). In addition, various reagents and kits for immobilization reactions are commercially available such as, for example, from Pierce Biotechnology.

In some embodiments, the PLIN1 polypeptide or fragment thereof is present in a tissue section, and the method comprises contacting a tissue section with a biological sample and a detectably-labeled secondary antibody under conditions in which a complex is formed between PLIN1 polypeptides in the tissue section, a corresponding PLIN1 autoantibody in the biological sample, if present, and the detectably-labeled secondary antibody; and (b) identifying a pattern of complex formation in the tissue sample by detecting the detectably-labeled secondary antibody, wherein the presence of a pattern of complex formation is indicative of the presence of PLIN1 autoantibodies in the biological sample, and wherein the absence of a pattern of complex formation is indicative of the absence of PLIN1 autoantibodies in the biological sample.

In some embodiments, the three components—the tissue section, the biological sample, and the detectably-labeled secondary antibody—are combined under conditions in which a complex is formed between PLIN1 polypeptides in the tissue section, and a corresponding PLIN1 autoantibody in the biological sample, if present, and the detectably-labeled secondary antibody. Using the detectable label and appropriate detection means, the pattern of complex formation within the tissue sections is identified. The pattern of complex formation within the tissue sections is directly related to the cellular location(s) of the antigen (e.g., an antigenic PLIN1 polypeptide) bound by an autoantibody, when present, in the biological sample. As described herein, the presence of a particular pattern of complex formation in one or more types of tissue indicates the presence of PLIN1 autoantibodies in the biological sample.

In instances where the PLIN1 polypeptide and antigenic fragment or variant thereof is in a phage display or eukaryotic cell display library, the presence of a PLIN1 autoantibody in a biological sample from a subject is assessed by contacting the biological sample with a phage display or eukaryotic cell display library. An appropriate display library includes a plurality of eukaryotic cells or phage that express a plurality of peptides including PLIN1 polypeptide fragments and variants thereof on the surface of the eukaryotic cells or phage. For example, the PLIN1 polypeptide fragments and variants thereof may be expressed as fusion proteins with a membrane protein on the surface of the eukaryotic cells or phage. Each cell or phage in the library expresses a different peptide. In some instances, the eukaryotic cell may be a yeast cell or a mammalian cell such as a human cell. The biological sample can be assayed to detect whether there is specific protein-protein interaction with any of the peptides expressed on the surface of the eukaryotic cells or phage. Methods of detecting protein-protein interactions using phage display are well-known in the art. For example, the putative immunogen may be bound to a solid support and the phage library applied thereto. After washing the solid support, any phage that remain bound to the solid support may express a PLIN1 autoantibody that can binding specifically to a PLIN1 polypeptide fragment or variant thereof. The phage DNA is isolated (after bacterial amplification) and sequenced to identify the sequence of the peptide expressed by the phage. Such peptides may then be further assessed individually for specific binding to the putative immunogen such as, for example, by immunoprecipitation, Western blot, or other immunoassay. In some instances, where the display library comprises eukaryotic cells, specific protein-protein interaction with any of the peptides may be assessed by flow cytometry. In some instances, the eukaryotic cells of the display library may be yeast cells. In some instances, the eukaryotic cells of the binding pool may be mammalian cells such as human cells. The peptides expressed on the cells of the display library may be fluorescently labeled (see discussion above regarding detectably-labeled secondary antibodies for exemplary fluorescent labels). The biological sample and the display library may be combined, and FACS analysis performed to identify cells that express peptides that are bound specifically to a PLIN1 autoantibody. In some instances, the identified cells may then be expanded in vitro, and the DNA or the RNA analyzed, such as by next generation sequencing. In some instances, single cell PCR may be performed followed by RNA and/or DNA sequence analysis. Other exemplary methods for assessing protein-protein interactions between a biological sample that contains a PLIN1 autoantibody and a display library include those described in Jardine, J., et al., 2013, Science 340(6133):711-716 and McGuire, A. T., et al., 2014, J. of Virology 88(5):2645-2657, both of which are incorporated by reference in their entireties herein. In one embodiment, the phage display system described in Example 1 may be used. See also WO 2020/190700, which is incorporated by reference in its entirety herein for the teaching of the phage display system.

In some instances, more than one of the detection methods described above may be used in a complementary manner for more reliable results. In some embodiments, other immunoassays can be performed either in alternative to or before and/or after the immunohistochemistry methods. For example, a Western blot may be performed using, for example, a panel of known antigens associated with autoantibodies, the panel including a PLIN1 polypeptide or antigenic fragment or variants thereof, the results of which may warrant further evaluation using, for example, the immunohistochemistry methods described herein. In another example, an immunohistochemistry method as described herein may be performed, followed by a Western blot in order to, for example, further confirm the specific antigens, including the PLIN1 polypeptide, recognized by the autoantibodies in the biological sample. In another example, a phage or eukaryotic cell display library that includes a plurality of eukaryotic cells or phage that express a plurality of peptides including PLIN1 polypeptide fragments and variants thereof on the surface of the eukaryotic cells or phage may be used to assess for the presence of PLIN1 autoantibodies in the biological sample from the subject, and then followed by a radioligand binding assay method or an immunohistochemistry method as described herein. In another example, the biological sample may be assessed by a radioligand binding assay method first, with confirmation by assessing the sample using a phage or eukaryotic cell display library.

Any data demonstrating the presence or absence of a PLIN1 autoantibody and the PLIN1 polypeptide or antigenic fragment or variant thereof may be correlated with reference data. For example, detection of a PLIN1 autoantibody indicates that the subject who provided the sample analyzed has or is at risk of developing lipodystrophy. If the subject has been previously diagnosed, the amount of PLIN1 autoantibodies detected at the time of prior diagnosis and in the present time may be correlated to find out about the progression of the disease and/or the success of a treatment. For example, if the amount of PLIN1 autoantibodies is found to increase, it may be concluded that the disease is progressing and/or that any treatment attempted is unsuccessful.

Kits and devices useful for performing the methods of Section A are described below in Section B.

B. Kits and Devices

In another aspect, provided in this disclosure are kits and panels containing one or more PLIN1 polypeptides or antigenic fragments or variants thereof to which PLIN1 autoantibodies can specifically bind. The polypeptide used in the kits and panels is preferably designed such that it is immunogenic, particularly that it binds to PLIN1 autoantibodies from subjects. In some instances, the kits include a panel as provided herein, such as a prognostic or diagnostic panel.

In certain embodiments, a kit as described herein includes one or more solubilizing agents for increasing the solubility of a polypeptide such as, for example, a buffer solution. The kit may further include reagents provide a detectable signal when used in conjunction with the PLIN1 polypeptides or fragments or variants thereof and a biological sample. In some embodiments, the kit includes a detectably-labeled secondary antibody that is able to bind to a PLIN1 autoantibody specifically binding to said one or more PLIN1 polypeptides or fragments or variants thereof. Reagents for the detection of the secondary antibody label can also be included in the kit. The secondary antibody is detected by a method that depends on a labeling group used. Exemplary labels for secondary antibodies are described above in this disclosure.

In addition, a kit can include directions for using the PLIN1 polypeptides or fragments or variants thereof and/or directions for practicing a method described herein; particularly, detecting PLIN1 autoantibodies in a biological sample. The concentration or amount of PLIN1 autoantibodies contained in the biological sample is indirectly measured by measuring the amount of the detectable label. The obtained measurement value may be converted to a relative or absolute concentration, amount, activity, etc. using a calibration curve or the like.

In some embodiments, a kit or a panel as provided herein includes a reference sample, such as a normal control sample. In some embodiments, a kit or a panel as provided herein includes one or more control antibody that detects an antigen that is expected to be present in a biological sample such as, for example, a biological sample from a healthy subject, or a biological sample from a subject with lipodystrophy. If such a sample is included, the obtained measurement values for such sample are compared with the results of the test sample, so that the presence or absence of lipodystrophy condition in the subject can be more objectively determined.

In addition to the one or more PLIN1 polypeptides, fragments, and/or variants, the panel can include additional polypeptides such as, for example, positive or negative controls or other antigens known to bind to autoantibodies of prognostic and/or diagnostic value, particularly those related to lipodystrophy and/or autoimmune disease.

In one aspect, provided herein is a prognostic or diagnostic device comprising a panel as described above, the panel including one or more PLIN1 polypeptides or antigenic fragments or variants thereof. In some embodiments, such a prognostic or diagnostic panel device comprises one or more PLIN1 polypeptides, fragments, or variants in a form as described above that allows contacting it with an aqueous solution, more preferably the liquid human sample, in a straightforward manner. In particular, the one or more PLIN1 polypeptides, fragments, or variants may be immobilized on the surface of a carrier, which carrier comprises, but is not limited to glass plates or slides, biochips, microtiter plates, beads, for example magnetic beads, chromatography columns, membranes or the like. Exemplary devices include line blots, microtiter plates and biochips. In some embodiments, the device can include additional polypeptides such as, for example, positive or negative controls or other antigens known to bind to autoantibodies of prognostic and/or diagnostic value, particularly those related to autoimmune diseases as discussed above.

C. Medical Methods

In one aspect, provided are methods of identifying early signs of autoimmune-related lipodystrophy in subjects who currently receive or have previously received ICI therapy. In some instances the subject receive or has previously received ICI therapy to treat cancer, such as, for example, melanoma. In some instances, the subject is monitored during the course of or after ICI therapy using the provided methods. For example, the methods provided herein may be used for early detection of a lipodystrophy condition before the manifestation of clinical symptoms. In some instances, the presence of PLIN1 autoantibodies may be used as an early indicator of autoimmune-related lipodystrophy. The provided methods are generally useful for detecting early evidence of lipodystrophy. In some instances, the presence of PLIN1 autoantibodies may predict the development of autoimmune-related lipodystrophy in a subject who currently receives or has previously received ICI therapy. In some instances, the presence of PLIN1 autoantibodies may indicate that the subject is at risk of developing autoimmune-related lipodystrophy due to ICI therapy. In some cases, ICI therapy in such patients may be altered or terminated in order to prevent the development or progression of lipodystrophy. In some cases, confirmation of lipodystrophy with the presence of PLIN1 autoantibodies may allow targeted treatment of the condition (e.g., with a peroxisome proliferator-activated receptor (PPAR) agonist; described further below) while allowing the subject to continue to be treated with the ICI therapy.

In some instances, a subject that currently receives or has previously received ICI treatment may develop an autoimmune disease. For example, the subject may have presented with immune-related adverse events (IRAE). In such cases, the provided methods are useful for monitoring the subject to detect early evidence of lipodystrophy or determine if the subject is at risk of developing autoimmune-related lipodystrophy.

In some instances, the provided methods are also useful for monitoring the immune response of seropositive patients in the course of ICI therapy. For example, the provided methods can be used to monitor the immune response of subjects that have been determined to have elevated PLIN1 polypeptide levels in the course of ICI therapy.

In another aspect, the provided methods can be used to determine the nature of a lipodystrophy condition that appears in the course of, or after, ICI therapy. In some instances, the provided methods can be used to determine if a diagnosed lipodystrophy is related to autoimmunity and/or caused by ICI therapy. For example, a subject that receives or has previously received ICI treatment may develop signs and symptoms associated with lipodystrophy. In some instances, the provided methods are useful for investigating symptoms that appear after ICI therapy and are not explainable by metastasis. In some cases, the subject presents with sign and symptoms of lipodystrophy (such as AGL or APL). Such symptoms may include one or more of insulin resistance, weight loss, low serum leptin, hyperphagia, polydipsia, dyslipidemia, acute pancreatitis, hepatic cirrhosis, proteinuria and renal failure, or polyuria. A subject exhibiting such symptoms may be assessed using the provided methods to determine if lipodystrophy is related to autoimmunity as indicated by detection of PLIN1 autoantibodies in the biological sample from the subject. If a PLIN1 autoantibodies are present, the ICI therapy may be terminated or the subject may receive additional treatment for the lipodystrophy. In some cases, the ICI therapy administered to the subject may be altered or replaced with a different therapy.

In some embodiments, the subject is also diagnosed with a metabolic abnormality (such as insulin resistance) and/or an autoimmune disease. The methods comprise detecting the presence of PLIN1 autoantibodies in a biological sample from the subject using an in vitro detection method, particularly methods using immunohistochemical detection of the PLIN1 autoantibodies. In some embodiments, a subject that is determined to be serologically positive for a PLIN1 autoantibody is diagnosed as having lipodystrophy. In some embodiments, the methods include a step of performing an examination of the subject to determine the presence of lipodystrophy in the subject. Any of the detection methods, kits, or devices discussed above in Sections A and B may be used.

In some instances, the provided methods are useful for screening a subject that is determined to be treated with ICI therapy and may be at risk of developing an autoimmune-related condition, such as lipodystrophy. In some instances, the subject may not be diagnosed with an autoimmune disorder but may be at risk of developing such a condition. In such cases, the provided methods may be useful as an indicator in determining if an ICI therapy is appropriate for the subject.

The presence (or absence) of a PLIN1 autoantibody in a biological sample from a subject that has been treated or is being treated with ICI therapy is detected using any of the methods, kits, or devices described in Sections A and B. Detection of the PLIN1 autoantibody in the biological sample indicates that the subject has autoimmune-related lipodystrophy. In some embodiments, an examination of the subject is performed to evaluate the presence of clinical symptoms of lipodystrophy. In some embodiments, the examination may be one or more of a body fat assessment (e.g., using caliper measurements of skinfold thickness, magnetic resonance imaging, or biphotonic absorptiometry), measurement of glucose, lipids, glycated hemoglobin (Ac1), and leptin levels, histology examination on biopsy tissue (e.g., liver tissue). See also e.g., Handelsman et al. (2013), “The clinical approach to the detection of lipodystrophy—an AACE consensus statement,” Endocrine Practice, 19 (1), 107-116.

In another aspect, provided are methods of treating a subject having one or more PLIN1 autoantibodies. In some instances, the provided methods are useful for treating patients that develop autoimmune-related lipodystrophy after ICI therapy. In some instances, the provided methods are useful for treating a subject that is at risk of developing lipodystrophy. In some embodiments, the method includes a step of detecting the presence or absence of a PLIN1 autoantibody in a biological sample from the subject. In some embodiments, the subject is seropositive for a PLIN1 autoantibody. The provided methods include a step of performing an examination of the subject to determine the presence of lipodystrophy in the subject or of associated conditions as described above in Section A, wherein the subject produces a PLIN1 autoantibody. In some cases, the patients may have recovered from cancer when they present with lipodystrophy or present with symptoms that indicate they may be at risk of developing lipodystrophy (e.g., metabolic abnormalities and/or other autoimmune diseases).

In some embodiments, the provided methods may include a step of a treatment for lipodystrophy to the subject. For example, the methods may include administering an immunosuppressive therapy and/or a peroxisome proliferator-activated receptor (PPAR) agonist to the subject. In some embodiments, the method includes (a) detecting the presence PLIN1 autoantibody that binds specifically to a PLIN1 antigenic polypeptide or fragment thereof in a biological sample from a subject, wherein detecting the PLIN1 autoantibody in the biological sample indicates that the subject has lipodystrophy or is at risk of developing lipodystrophy, and administering to the subject an immunosuppressive therapy and/or a peroxisome proliferator-activated receptor (PPAR) agonist.

In some embodiments, immunosuppressive therapy is administered to the subject to treat the lipodystrophy. In such instances, administration of the ICI therapy to the subject is stopped while the treatment for the lipodystrophy is administered. Immunosuppressive therapies are therapies that lower the activity of the body's immune system. Such therapies are useful to treat conditions in which the immune system is overactive, such as autoimmune diseases. The immunosuppressive therapy administered to the subject may include at least one of an immunosuppressant drug, intravenous immunoglobulin administration, plasma exchange plasmapheresis, or immunoadsorption. For example, first-line immunosuppressive therapy can include corticosteroids, intravenous immunoglobulin, plasma exchange plasmapheresis, and immunoadsorption. In some instances, second-line immunosuppressive therapy may be need to be administered. Exemplary second-line immunosuppressive therapy is treatment with immunosuppressant drugs such as rituximab and cyclophosphamide.

Immunosuppressant drugs include corticosteroids (such as prednisone, budesonide, prednisolone), Janus kinase inhibitors (such as tofacitinib), calcineurin inhibitors (such as cyclosporine, tacrolimus), mTOR inhibitors (such as sirolimus, everolimus), IMDH inhibitors (such as azathioprine, lefunomide, mycophenolate), monoclonal antibodies (such as basiliximab, daclizumab, muromonab, adalimumab, certolizumab, etanercept, golimumab, infliximab, ixekizumab, natalizumab, rituximab, secukinumab, tocilizumab, ustekinumab, vedolizumab), and other biologics (such as abatacept, anakinra). Therapeutic targets for these immunosuppressant drugs include B cells and short-lived plasma cells (rituximab) and specific cytokines associated in the autoimmune and inflammatory process (tocilizumab and low-dose interleukin (IL)-2). Antiproliferative agents targeting lymphocyte proliferation (cyclophosphamide, azathioprine, mycophenolate mofetil, etc.) can also be used in refractory cases or to maintain remission.

In some instances, the subject is administered an intravenous immunoglobulin (IVIg), which is a blood product extracted from the collected pool of plasma from over a thousand donors. IVIg provides antibodies to a broad range of pathogens, and is used to provide passive immunity for patients with immunodeficiency. In some instances, IVIg is administered as a monotherapy. In some instances, after or in combination with high-dose steroids, or with plasma exchange plasmapheresis, rituximab, or other immunotherapeutic agents.

Plasma exchange plasmapheresis (also referred to as PLEX) may also be used. PLEX removes autoantibodies and other pathologic substances in the plasma. PLEX also alters the immune system by changing lymphocyte numbers and their distribution, T-suppressor cell function, and T-helper cell phenotypes. Steroids alone can be insufficient to ameliorate autoantibody-mediated immune process, and direct removal or neutralization of autoantibodies from the circulation by PLEX and IVIg may show a synergistic effect. In addition, PLEX increases the proliferation of antibody-producing cells and this could increase susceptibility of these cells to immunosuppressants and chemotherapeutic agents. Immunoadsorption is a refined form of PLEX that enables the selective removal of Igs from separated plasma through high-affinity adsorbers (e.g., Protein A).

In some instances, the subject can be treated with an immunosuppressive therapy comprising oral administration of PLIN1 polypeptide or immunogenic fragments thereof. Such therapy is termed oral tolerization and involves feeding to the subject the autoantigen inducing in order to suppress the immune response by invoking oral tolerance.

In some embodiments, a peroxisome proliferator-activated receptor (PPAR) agonist is administered to the subject to treat the lipodystrophy. In such instances, the subject may continue to receive ICI therapy while also being administered a PPAR agonist. PPAR agonists that may be used include, for example, PPAR-alpha agonists (such as clofibrate, gemfibrozil, bezafibrate, and fenofibrate), PPAR-gamma agonists (such as thiazolidinediones (TZDs; e.g., pioglitazone, rosiglitazone, and lobeglitazone), PPAR-delate agonists (such as GW501516), and dual PPAR agonists, also known as glitazars (such as saroglitazar, aleglitazar, muraglitazar and tesaglitazar). In some embodiments, the method includes administering pioglitazone to the subject to treat the metabolic conditions associated with lipodystrophy.

In some embodiments, the provided methods comprise stopping administration of the ICI therapy to the subject and administering an immunosuppressive therapy and a PPAR agonist to the subject to treat the lipodystrophy.

In some instances, treatment of the subject includes reducing the amount of PLIN1 autoantibodies in the subject. For example, the amount of PLIN1 autoantibodies may be reduced by plasmapheresis, which involves the removal of blood from the subject, separation of the plasma from blood cells, and reinjection of the cells. In particular embodiments, plasmapheresis may be carried out by plasma exchange, in which the plasma removed is replaced in whole or in part by a plasma substitute such as lactated Ringer's solution, or by plasma perfusion, in which the autoantibodies are separated from the plasma and the plasma then returned to the subject.

Autoantibodies may be removed from the subject's plasma by any suitable technique, such as by contacting the plasma to a solid support having an immunoglobulin binding protein (e.g., protein A) immobilized thereon), removing the plasma from the solid support, and then returning the plasma to the subject. Typically, this method is practiced by immobilizing the immunoglobulin binding protein on an affinity support in an affinity column, passing the blood plasma through the affinity column, and returning the plasma to the subject (optionally, but preferably, recombining the plasma with the patient's blood cells). The method may also be carried out on whole blood or other suitable blood fraction, which may then be recombined with blood cells and then returned to the patient, as will be appreciated by persons skilled in the art.

In a variation of the foregoing, the PLIN1 protein or one or more antigenic fragments thereof may be immobilized on the solid support, and the PLIN1 autoantibodies selectively removed from the subject's blood by contacting the subject's blood, blood plasma, or other suitable fraction to the solid support, as described above. Such procedures advantageously avoid substantial reduction in levels of other antibodies in the subject undergoing treatment.

In many instances, following treatment, the subject is monitored at regular intervals over time (such as biannually, annually, or every two to five years) for indications of PLIN1 autoantibody recurrence.

In accordance with the present disclosure, there may be employed conventional molecular biology, microbiology, biochemical, and recombinant DNA techniques within the skill of the art. Such techniques are explained fully in the literature. The provided methods and compositions will be further described in the following examples, which do not limit the scope of the methods and compositions of matter described in the claims.

### EXAMPLES

### Example 1. Related Studies on PLIN1 Autoantibodies Associated with Lipodystrophy

The inventors of the methods provided in this disclosure conducted studies identifying an association between PLIN1 autoantibodies and lipodystrophy generally. These studies are described in co-filed International Application No. PCT/US2020/067190, filed Dec. 28, 2020, which is incorporated herein by reference in its entirety for all purposes. The inventors of the methods provided in this disclosure also conducted studies identifying an association between PLIN1 autoantibodies and lipodystrophy in patients undergoing checkpoint immunotherapy (CP). These studies are described in Mandel-Brehm et al. (2021), “Perilipin-1 autoantibodies linked to idiopathic lipodystrophy in the setting of two distinct breaks in immune tolerance,” preprint in medRxiv, Sep. 28, 2021, doi:10.1101/2021.09.24.21263657, which is incorporated herein by reference in its entirety for all purposes. These studies are described here.

Phage-Display and Immunoprecipitation protocol (PhIP-Seq): The T7 Phage Display library (PhIP-Seq library) used in this study was validated previously (O'Donovan et al. (2020), “High-resolution epitope mapping of anti-Hu and anti-Yo autoimmunity by programmable phage display,” Brain Communications, 2 (2), fcaa059) and can be accessed at github.com/derisilab-ucsf/PhIP-PND-2018. The experiment is performed as detailed in Mandel-Brehm et al. (2019; “Kelch-like Protein 11 Antibodies in Seminoma-Associated Paraneoplastic Encephalitis,” New England Journal of Medicine, 381(1), 47-54) and Vazquez et al. (2020; “Identification of novel, clinically correlated autoantigens in the monogenic autoimmune syndrome APS1 by proteome-wide PhIP-Seq,” eLife, 9: e55053), both of which are incorporated by reference in their entirety herein. PhIP-Seq methods are also described in WO 2020/190700, which is incorporated by reference in its entirety herein. However, instead of one microliter of human sera per immunoprecipitation (IP), one microliter of sera from mice was used in the Examples herein. Four AIRE knockouts and three age-matched wildtype controls were tested. Briefly, mouse sera were mixed with one milliliter of PhIP-Seq library (10\hat{ }10 pfu/mL) and incubated for 12-18 hours at 4° C. Antibody-bound phage were immunoprecipitated using a mix of protein A and protein G magnetic beads (Thermo Fisher), eluted and DNA sequenced to identify the unknown phage antigen(s). First, DNA sequencing reads were converted to “peptide reads” by aligning to the reference library and total reads were normalized to reads per 100,000 reads (RP100K) for each sample. Then the total number of peptide reads was summed with respect to annotated proteins (20,000 unique proteins represented in the library). The criteria for candidate antigens were an RP100K greater than 50, present in greater than two AIRE knockout (“KO”) mice, and not present in any wildtype mice. Putative antigens discovered by phage display were validated in two orthogonal assays including a 293T cell overexpression system and mouse brain immunohistochemistry described below.

Validation of PLIN1 antibodies using 293T expression system: Full-length mouse PLIN1 ORF clone was purchased from Origene (Human: Cat No. RC206292; Mouse: Cat. No. MR208288). For this clone, transcription is driven from a CMV6 promoter and the expressed protein is Myc-Flag tagged The validation protocol including 293T cell transfections with plasmid, whole cell lysate preparation, immunoprecipitations with sera and/or commercial antibodies and western blotting as previously described. See e.g., Mandel-Brehm et al, 2019 (supra) and WO 2020/190700. Whole cell lysates containing overexpressed PLIN1 were made to 1 mg/ml, and 10 microliters was set aside prior to IP for input for western blotting. The seven original archived samples (WT n=3, AIRE KO n=4) and an additional AG bead only control (no mouse sera added to IP) were tested for the ability to IP PLIN1 from the whole cell lysate. One microliter of mouse sera or nothing (mock) was added to 500 microliters of whole cell lysate, incubated for 12 hour at 4° C. Antibodies were immunoprecipitated with protein A and protein G beads (as done in PhIP-seq), washed three times with RIPA buffer (140 mM NaCl, 10 mM Tris-HCL, 1.0% Triton-X, 0.1% SDS), and boiled in 2× Laemmli buffer. Entire IP elutions were electrophoresed on a 4-12% Bis-Tris protein gel (Thermo Fisher, NP0349BOX), transferred to 0.45 micron nitrocellulose, and immunoblotted with primary anti-Flag antibody (Rabbit anti-Flag, 1:5000, Cell Signaling Technology) and infrared secondary antibody Goat anti-Rabbit IgG-IR800 (LICOR 926-68703).

Discovery of autoantibodies to PLIN1 in mice, motivating testing of PLIN1 in human syndromes: Autoantibodies to PLIN1 were first identified by screening sera from murine knockouts of the Aire gene (Aire −/−). Mutations in AIRE in humans leads to the development of Autoimmune Polyglandular Syndrome type 1 (APS1/APECED; OMIM #240300), which is characterized by multi-organ autoimmunity (REF) and production of high affinity antibodies to tissue-restricted antigens. Mice lacking the AIRE gene (AIRE KO) recapitulate the heterogeneous antibody response and can inform new biology of APS1, and broader human autoimmune diseases. Idiopathic antigens in previously banked Aire −/− sera were investigated using PhIP-Seq, a proteome-wide antigen discovery method (Larman et al 2011, see methods). Four Aire −/− as well as three age-matched wildtype controls (Aire +/+) were tested. Aire −/− mean RP100Ks were plotted against Healthy mean RP100Ks for each protein in the library (n=100). A candidate antigen was any protein with a positive cutoff of 150 RP100K, that was present in two or more Aire −/− mice and not present in wild-type controls (Aire +/+). Although each sample exhibited distinct patterns of PhIP-Seq reactivity, only one protein, PLIN1, satisfied this stringent criteria. Peptides contributing to the PhIP-Seq signal for PLIN1 in the four AIRE knockouts are shown in Table 1. Antibody reactivity to full-length PLIN1 in three of four Aire −/− (100% concordance with PhIP-Seq) was validated using 293Tcell overexpression assay and IP elutions from whole cell lysates. Because PLIN1 demonstrates subcutaneous fat-specific expression PLIN1-reactive mouse serum was also tested on sections of fat. It was confirmed that sera from Aire −/− mice are reactive to tissue containing fat and further that mouse antibody reactivity co-localizes with reactivity of commercial antibody to PLIN1.

TABLE 1
Peptides contributing to the PhIP-Seq signal
for PLIN1

| | | SEQ |
|---|---|---|
| | | ID |
| Fragment | Amino acid sequence | NO |
| 1 | LQLPVVSGTCECFQKTYTSTKEAHPLVASVCNAY | 2 |
| | EKGVQSASSLAAWSM | |
| 2 | PLVASVCNAYEKGVQSASSLAAWSMEPVVRRLST | 3 |
| | QFTAANELACRGLDH | |
| 11 | ASVAMQAVSRRRSEVRVPWLHSLAAAQEEDHEDQ | 4 |
| | TDTEGEDTEEEEELE | |
| 4 | HLEEKIPALQYPPEKIASELKDTISTRLRSARNS | 5 |
| | ISVPIASTSDKVLGA | |
| 5 | STRLRSARNSISVPIASTSDKVLGAALAGCELAW | 6 |
| | GVARDTAEFAANTRA | |

PLIN1 expression in mouse and human thymus: To evaluate whether PLIN1 exhibits Aire-dependent thymic expression, a publicly available bulk RNA-seq dataset previously published by Sansom et al. 2014) was assessed. The relative expression of HMI in medullary thymic epithelial cells (mTECs) isolated from Aire−/− was compared to Aire +/+ mice. PLIN1 expression was substantially reduced in the Aire−/− mTECs, similar to the expression patterns exhibited by known Aire-dependent genes Inst, Cyp11a1, and Nlrp5. In view of this data, further candidate exploration of PLIN1 autoantibodies in human lipodystrophies was performed. A well-established Radioactive Ligand Bind Assay was adapted to screen for autoantibodies to PLIN1 in humans as described in Example 2.

### Example 2. Approach and Methods for PLIN1 Autoantibody Assessment

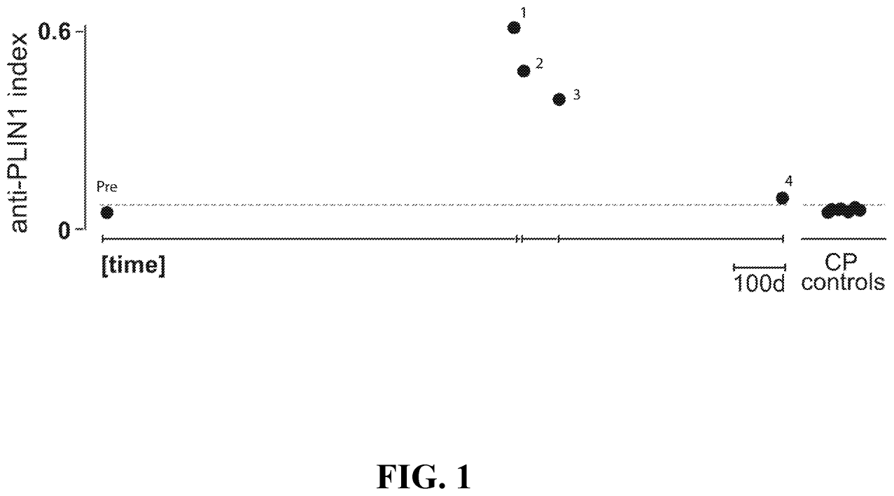

Radioligand binding assay (RLBA): An expression plasmid containing full-length human PLIN1 coding sequence under the control of a T7 promoter (Origene, RC206292) was sequence verified and used as a DNA template for in vitro translation of the PLIN1 protein. PLIN1 was synthesized in the presence of ³⁵S-Methionine to radiolabel the protein as previously described (Berson, et al. (1956), “Insulin-I131 metabolism in human subjects: Demonstration of insulin binding globulin in the circulation of insulin treated subjects,” Journal of Clinical Investigation, 35(2), 170-190; Shum et al. (2009), “Identification of an autoantigen demonstrates a link between interstitial lung disease and a defect in central tolerance,” Science Translational Medicine, 1(9), 9ra20; Vazquez et al. (2020), “Identification of novel, clinically correlated autoantigens in the monogenic autoimmune syndrome APS1 by proteome-wide PhIP-Seq,” eLife, 9: e55053). Serum samples from the patient were collected at five different time points throughout her clinical course (pre-therapy, post-therapy-1, post-therapy-2, post-therapy-3, and post-therapy-4) under informed consent. Radioligand binding assays screening for PLIN1 antibodies was performed on sera the different time points from the patient with AGL following cancer immunotherapy as well as on sera from checkpoint inhibitor treated control patients without lipodystrophy (n=7) and healthy controls (n=11). All sera were incubated with radiolabeled PLIN1 at 4° C. overnight. Antibodies were then immunoprecipitated with protein A/G beads, washed, and total radioactive counts were obtained by scintillation. A known PLIN1-specific antibody (Sigma-Aldrich, #HPA024299) was used as a positive control. The Antibody index was calculated as follows: (sample value−mean blank value)/(positive control antibody value−mean blank value). A cut-off for a positive result was set as 3 standard deviations above the mean for normal controls (indicated by dotted line in FIG. 1).

Indirect immunofluorescence on mouse enteric tissue: Adult wild-type mice were sacrificed, then perfused with 4% paraformaldehyde, and the stomach was post fixed for 1 hour, followed by sucrose/OCT embedding for cryosectioning. Sections were cut 12 micron thick. Indirect immunofluorescent stains were obtained by incubating serum from the patient sample or control sample at a dilution of 1:1000 on tissue section. Sera was used from either a control (immunotherapy but no AGL) or the AGL patient (Immunotherapy with AGL) from various time points. The time points for the control group with no autoimmunity were checkpoint pre-treatment and checkpoint post treatment. The time points for the patient were: pre-therapy, post-therapy-1, post-therapy-3, and post-therapy-4. Samples were washed and then developed with a FITC-conjugated secondary anti-human IgG antibody (Abcam). Images were captured using a Nikon Ti confocal microscope at the UCSF imaging core. Colocalization was assessed qualitatively through visual inspection by experimenter blind to study.

### Example 3. Clinical Phenotyping of Patient—Case Report

A patient, described in Jehl et al. 2019, “Acquired Generalized Lipodystrophy: A new cause of anti-PD-1 immune-related diabetes, Diabetes Care, 42 (10):2008-2010) developed severe lipoatrophy, low serum leptin, and high insulin levels after receiving immune checkpoint blockade therapy with nivolumab for treatment of recurrent, metastatic melanoma. The patient presented at the age of 62 with the development of severe hyperglycemia and lipoatrophy. The patient had been previously diagnosed with melanoma at the age of 50 in her shoulder, which was treated with surgery. Ten years later she experienced mental status changes, and metastatic melanoma was detected in her brain, lung, and liver that was positive for the V600E BRAF somatic mutation. The patient was then initiated on nivolumab therapy with a partial tumor response. Sixteen months after treatment was initiated, the patient presented with progressive weight loss and elevated liver function tests (LFTs). Workup included a liver biopsy, which revealed severe steatosis, and then nivolumab treatment was halted. A month later the patient developed further weight loss, hyperphagia, polydipsia, and polyuria. Type 1 diabetes-related autoantibodies (anti-GAD, IA2, and ZnT8) were negative and fasting plasma insulin, C-peptide, HOMA of insulin resistance were all consistent with insulin resistant diabetes. Her physical exam was notable for severe loss of subcutaneous fat in a broad distribution with muscle prominence consistent with lipodystrophy. Laboratory testing was notable for an undetectable leptin level and elevated triglycerides. Clinical testing for 23 lipodystrophy associated genetic variants was also negative (AKT2, BSCL1, BSCL2, CAV1, CIDEC, DYRK1B, INSR, LIPE, LMF1, LMNA, LMNB2, NSMCE2, PCYT1A, PIK3R1, PLD3, PLIN1, POC1A, POLD1, PPARG, PSMB8, PTRF, TBC1D4, ZMPSTE24). A diagnosis of acquired generalized lipodystrophy was made and her diabetes was treated with metformin, dietary changes and high dose basal-bolus insulin. Four months following cessation of nivolumab, there was improvement in the patient's diabetes as noted by decreased insulin requirements and partial improvement in hypertriglyceridemia. Serum samples for the patient were collected at different time points throughout her clinical course under informed consent.

### Example 4. Results

Antibody reactivity: Sera from the patient at different time points was tested for autoantibodies specific to PLIN1 via RLBA. It was found that prior to treatment with immunotherapy, the patient was negative for PLIN1 autoantibodies. Following 34 courses of treatment with immunotherapy (nivolumab), the patient's serum contained autoantibodies to PLIN1 (FIG. 1). Notably, autoantibodies to PLIN1 appeared concurrently with the onset of lipodystrophy in this patient. To validate the immunoreactivity, immuno-labeling of mouse enteric tissue with patient sera and commercial antibody to PLIN1 colocalized in samples corresponding to post-treatment and lipodystrophy diagnosis timepoints, but not pre-treatment samples (see FIG. 3B in Mandel-Brehm et al., 2021, supra). Reactivity to PLIN1 appeared to decrease by 19 months after cessation of nivolumab, and although the patient's generalized lipodystrophy phenotype did not resolve their metabolic profile did improve.

1. A method of identifying a subject that is at risk of developing autoimmune-related lipodystrophy, (ii) identifying early evidence of autoimmune-related lipodystrophy in the subject, or (iii) detecting the presence of a Perilipin-1 (PLIN1) autoantibody in a biological sample from the subject, the method comprising:
(a) contacting the biological sample from the subject with a PLIN1 antigenic polypeptide, or a fragment thereof; and
(b) detecting the presence of binding of the PLIN1 antigenic polypeptide or fragment thereof to PLIN1 autoantibody in the biological sample,
wherein the subject has been previously treated or is being treated with immune checkpoint inhibitor (ICI) therapy.

2-3. (canceled)

4. The method of claim 1, wherein,
in (i), the presence of the binding of the PLIN1 antigenic polypeptide or fragment thereof to the PLIN1 autoantibody in the biological sample indicates that the subject is at risk of developing the autoimmune-related lipodystrophy, or
in (iii), the subject is suspected to have lipodystrophy or is diagnosed with lipodystrophy.

5. (canceled)

6. The method of claim 1, wherein the presence of the binding of the PLIN1 antigenic polypeptide or fragment thereof to the PLIN1 autoantibody in the biological sample indicates that the subject has autoimmune-related lipodystrophy.

7. The method of claim 1, wherein the subject has autoimmune-related lipodystrophy, and wherein the PLIN1 antigenic polypeptide or fragment thereof binds to said PLIN1 autoantibody in the biological sample.

8. The method of claim 1, wherein
the PLIN1 antigenic polypeptide or fragment thereof is heterologously expressed on the surface of a cell, a phage or a virus,
the PLIN1 antigenic polypeptide or fragment thereof is expressed in a phage display or eukaryotic cell display library,
the PLIN1 antigenic polypeptide or fragment thereof is an isolated, purified antigenic polypeptide or fragment thereof, and/or
the PLIN1 antigenic polypeptide or fragment thereof is an isolated, purified antigenic polypeptide or fragment thereof that is immobilized on a solid carrier.

9-11. (canceled)

12. The method of claim 1, wherein step (b) of detecting is performed by at least one of immunoprecipitation, microarray analysis, enzyme-linked immunosorbent assay (ELISA), or Western blot analysis.

13. The method of claim 1, wherein the PLIN1 polypeptide or fragment thereof comprises one or more of the sequences of SEQ ID NOs: 2-6.

14. The method of claim 1, wherein the PLIN1 antigenic polypeptide comprises the sequence of SEQ ID NO:1.

15. The method of claim 1, wherein the biological sample is serum, plasma, blood, or urine.

16. The method of claim 1, wherein the autoimmune-related lipodystrophy is acquired generalized lipodystrophy (AGL) or acquired partial lipodystrophy (APL).

17. The method of claim 1, wherein the subject has one or more of insulin resistance, low serum leptin, hyperphagia, polydipsia, dyslipidemia, acute pancreatitis, hepatic cirrhosis, proteinuria and renal failure, polyuria, type II diabetes, hypertriglyceridemia, hepatic steatosis, steatohepatitis, acanthosis nigricans, polycystic ovarian syndrome (PCOS), cardiovascular disease, or eruptive xanthomas, or one or more autoimmune disease.

18-19. (canceled)

20. The method of claim 17, wherein the autoimmune disease is dermatomyositis, polymyositis, Sjogren's syndrome, rheumatoid arthritis, psoriatic arthritis, ankylosing spondylitis, scleroderma, systemic sclerosis, or systemic lupus erythematosus.

21. The method of claim 1, wherein the subject has cancer or has had cancer.

22. A method of treating a subject having early evidence of lipodystrophy, the method comprising:
(a) detecting the presence PLIN1 autoantibody that binds specifically to a PLIN1 antigenic polypeptide or fragment thereof in a biological sample from a subject, wherein detecting the PLIN1 autoantibody in the biological sample indicates that the subject has lipodystrophy; and
(b) administering to the subject an immunosuppressive therapy and/or a peroxisome proliferator-activated receptor (PPAR) agonist,
wherein the subject has been previously treated or is being treated with immune checkpoint blockade therapy.

23. The method of claim 22, wherein the subject has symptoms of acquired generalized lipodystrophy (AGL) or acquired partial lipodystrophy (APL).

24. A method of treating a subject that is at risk of developing lipodystrophy, the method comprising:
(a) detecting the presence or absence of PLIN1 autoantibody in a biological sample from a subject using the method of claim 1, wherein detecting the PLIN1 autoantibody in the biological sample indicates that the subject is at risk of developing lipodystrophy; and
(b) administering to the subject an immunosuppressive therapy and/or a peroxisome proliferator-activated receptor (PPAR) agonist,
wherein the subject has been previously treated or is being treated with immune checkpoint blockade therapy.

25. The method of claim 24, wherein the subject has one or more of insulin resistance, low serum leptin, hyperphagia, polydipsia, dyslipidemia, acute pancreatitis, hepatic cirrhosis, proteinuria and renal failure, polyuria, type II diabetes, hypertriglyceridemia, hepatic steatosis, steatohepatitis, acanthosis nigricans, polycystic ovarian syndrome (PCOS), cardiovascular disease, or eruptive xanthomas.

26. (canceled)

27. The method of claim 24, wherein the subject has an autoimmune disease.

28. The method of claim 27, wherein the autoimmune disease is dermatomyositis, polymyositis Sjogren's syndrome, rheumatoid arthritis, scleroderma, systemic sclerosis, and systemic lupus erythematosus.

29. The method of claim 24, wherein the immunosuppressive therapy comprises at least one of an immunosuppressant drug, intravenous immunoglobulin administration, plasma exchange plasmapheresis, immunoadsorption, or administration of the antigenic PLIN1 polypeptide or immunogenic fragments thereof.
